(19) United States
(12) Patent Application Publication
(10) Pub. No.: US 2007/0203515 A1
(43) Pub. Date: Aug. 30, 2007

(54) CATHETER SYSTEM FOR CONNECTING ADJACENT BLOOD VESSELS

(76) Inventors: Richard R. Heuser, Phoenix, AZ (US); James D. Joye, Saratoga, CA (US)

Correspondence Address: KOLISCH HARTWELL, P.C., 200 PACIFIC BUILDING, 520 SW YAMHILL STREET, PORTLAND, OR, 97204, US

(21) Appl. No.: 11/735,350

(22) Filed: Apr. 13, 2007

Related U.S. Application Data
(63) Continuation-in-part of application No. 11/340,324, filed on Jan. 25, 2006.
(60) Provisional application No. 60/887,277, filed on Jan. 30, 2007.

(51) Int. Cl. A61B 17/32

(52) U.S. Cl. 606/184

(57) ABSTRACT

An apparatus is provided for assisting in creating a fistula between a first blood vessel having a first diameter and a second blood vessel. The apparatus includes a catheter with a resizable portion disposed near the distal end of the catheter. The catheter is traversed through the first vessel to a position adjacent to a location intended for a fistula connecting the first vessel to the second vessel. The resizable portion in a nominal configuration has a diameter which is less than the first diameter, allowing traversal through the first vessel. The resizable portion is manipulable to an active configuration having a diameter substantially equal to the first diameter, stabilizing the catheter within the first vessel. The resizable portion may be at least partially radiopaque, and may cover all but a free arc of the outer surface, allowing an unobstructed path for a piercing tool to pass.

### CROSS REFERENCE TO RELATED APPLICATIONS

This application is a continuation-in-part of application Ser. No. 11/340,324, filed Jan. 25, 2006, the disclosure of which is incorporated by reference. This application also claims the benefit of Provisional Application Ser. No. 60/887,277, filed on Jan. 30, 2007, the disclosure of which is incorporated by reference.

### BACKGROUND

This disclosure relates generally to a catheter system for connecting adjacent blood vessels, e.g, an artery and an adjacent vein to adapt the vein for arterial blood flow. More particularly the disclosure concerns a system of two catheters with mating, magnetic tips for creating openings in the artery wall and vein wall to form a fistula connecting the blood vessels. Another aspect of the disclosure provides for an apparatus to stabilize and position a catheter inside the lumen of a blood vessel.

A catheter apparatus and method for arterializing a section of a vein to bypass a clogged artery are shown in U.S. Pat. No. 6,464,665, which is hereby incorporated by reference. The method is used to bypass a stenosis in the artery that obstructs blood flow in a portion of the artery. If the obstructed portion of the artery can be bypassed, blood flow will be restored downstream from the stenosis. A vein running alongside the artery in the obstructed portion of the artery can be used for the bypass.

The catheter apparatus includes one catheter for inserting into the artery and another catheter for inserting into the adjacent vein. The physician maneuvers the tips of both catheters to coincident positions within each blood vessel adjacent one end of the obstructed portion of the artery. The physician then creates an opening from the inside of one blood vessel through the vessel wall and then through the wall of the other blood vessel.

A difficulty with this procedure is in co-locating the openings in the two blood vessels and holding the vessel walls in place to ensure that a channel will be created between the vessels so that blood will flow from one vessel to the other. Another issue is in keeping the catheters steady inside the vessels. In particular, veins often have diameters much larger than adjacent arteries, which allow a catheter too much freedom of movement inside the vein. Further, hitting a smaller artery from a larger vein with a needle can be difficult.

### SUMMARY OF THE DISCLOSURE

A first aspect of the disclosed system and method provides for creating paired, co-located openings and a consequent fistula between an artery and an adjacent vein to bypass an arterial blockage. The system includes a piercing tool on a first catheter that mates with a receptor on a second catheter to create the co-located openings at one side of the blockage. Magnets incorporated in either or both catheters may be used to draw the piercing tool into the receptor. The piercing tool and receptor typically are provided with complementary, mating contours to draw the piercing tool sufficiently into the receptor to ensure completion of the openings. The openings may be expanded by balloon angioplasty and a stent is typically then installed to interconnect the openings to ensure a fistula is established between the vessels. The process may be repeated at the other side of the arterial blockage to complete the bypass.

Another aspect of the disclosure provides for a catheter stabilizing and positioning apparatus including a catheter for inserting into a blood vessel with a resizable portion disposed on the outer surface of the catheter. A physician maneuvers the tip of the catheter to a position within a first blood vessel adjacent to the portion of the first blood vessel in which the physician intends to create a fistula to an adjacent blood vessel. The physician then expands the resizable portion to a diameter substantially equal to that of the first blood vessel. The physician may thereafter independently rotate the catheter apparatus within the first blood vessel in order to aim a piercing tool towards the location in which the physician will be making the fistula. The resizable portion may cover all but a first arc of the surface of the catheter free to allow uninhibited passage of the piercing tool.

### BRIEF DESCRIPTION OF THE DRAWINGS

FIG. 1 is a partial cross-sectional view showing an obstructed artery, including the obstruction and the area adjacent both ends of the obstruction, and a vein alongside the artery.

FIG. 2 is a cross-sectional view of an embodiment of the present disclosure in the blood vessels of FIG. 1 with a first catheter with a distal end inserted into the artery and a second catheter with a distal end inserted into the vein, the catheters carrying at their distal ends mating tips, i.e., a piercing tool on the first catheter and a receptor on the second catheter.

FIG. 3 is a cross-sectional view of the vein, artery, and two catheters, as in FIG. 2 with the tips of the catheters mated to create a pair of co-located openings in the walls of the vein and artery for connection of a fistula between the artery and the vein.

FIG. 4 is a cross-sectional view of the vein and artery with a balloon inserted through both openings.

FIG. 5 is a cross-sectional view of the vein and artery with a stent installed through the openings between the vein and artery to maintain a fistula therebetween.

FIG. 6 is a cross-sectional view of a first catheter inserted in the artery and a second catheter inserted in the vein at the other end of the obstruction depicted in FIGS. 1-4, the catheters including mating tips shown in a joined position to create a second pair of co-located openings through the vein and artery walls.

FIG. 7 is a cross-sectional view of the vein and artery with a balloon inserted through the second pair of openings between the vein and the artery.

FIG. 8 is a cross-sectional view of the vein and artery with a second stent installed through the second pair of openings between the vein and artery to maintain a fistula therebetween.

FIG. 9 is a close-up perspective view of the mating tips of the first and second catheters, showing the receptor, which includes a proximal end, a distal opening, and a channel providing a guide surface, and the piercing tool, which includes a needle and a plug encompassing the catheter adjacent the base of the needle, and showing the contours of the plug, needle, and receptor channel that provide for mating between the tips.

FIG. 10 is a piercing tool for use in a second embodiment of the present disclosure that includes a base and a needle that is offset from the base by an angle.

FIG. 11 illustrates the use of the piercing tool of FIG. 10 in conjunction with a double-balloon catheter to create openings in a vein and an artery.

FIG. 12 illustrates the use of the piercing tool of FIGS. 2, 3, 6, and 9 in conjunction with a double-balloon catheter to create openings in a vein and an artery.

FIG. 13 depicts a stabilizing apparatus disposed on a catheter outside a living body, shown with the resizable portion in its nominal configuration.

FIG. 14 depicts the embodiment of FIG. 13, shown in the lumen of a first vessel with its resizable portion in its active configuration.

FIG. 15 depicts the embodiment of FIGS. 13 and 14, with a piercing tool extending from the catheter into a second vessel.

FIGS. 16 and 16A are views of two similar but alternative embodiments according to the present disclosure, with the resizable portions shown in active configuration.

FIG. 17 is a cross-sectional view of the embodiment shown in FIGS. 16 and 16A.

FIG. 18 shows an alternative embodiment of the present disclosure in the lumen of a first vessel, with the resizable portion shown in active configuration.

FIG. 19 shows the device of FIG. 18 viewed from the plane defined by arrows 19 of FIG. 18.

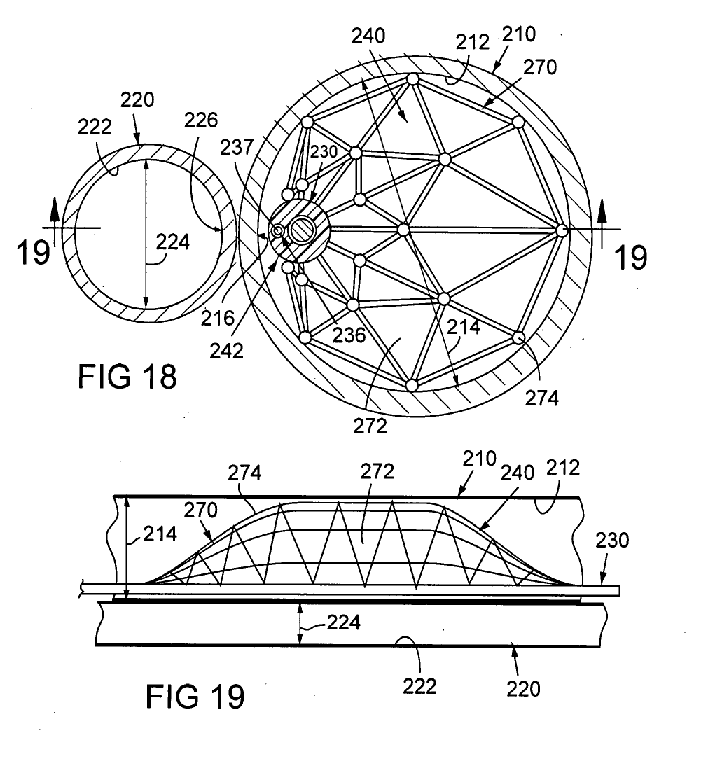

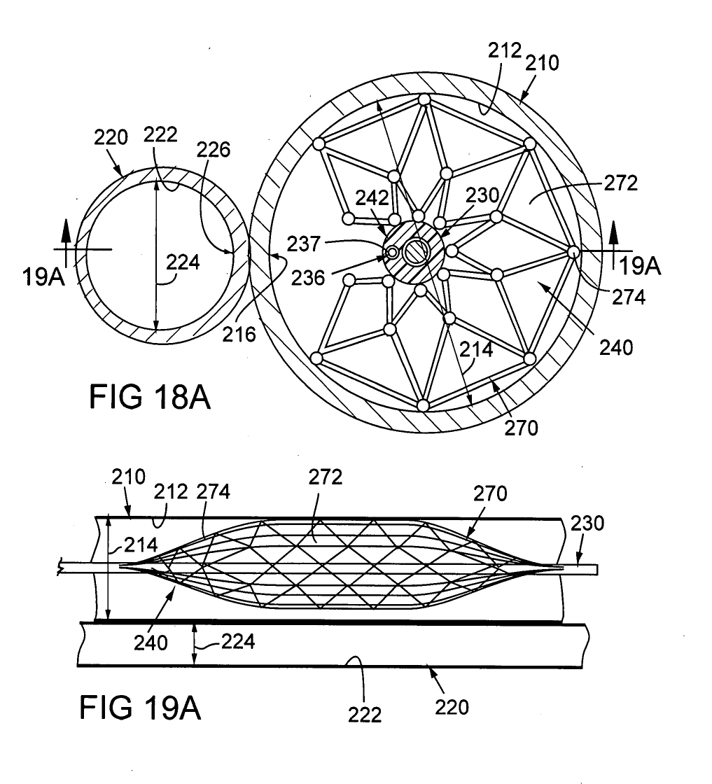

FIG. 18A shows an alternative embodiment of the present disclosure in the lumen of a first vessel, with the resizable portion shown in active configuration.

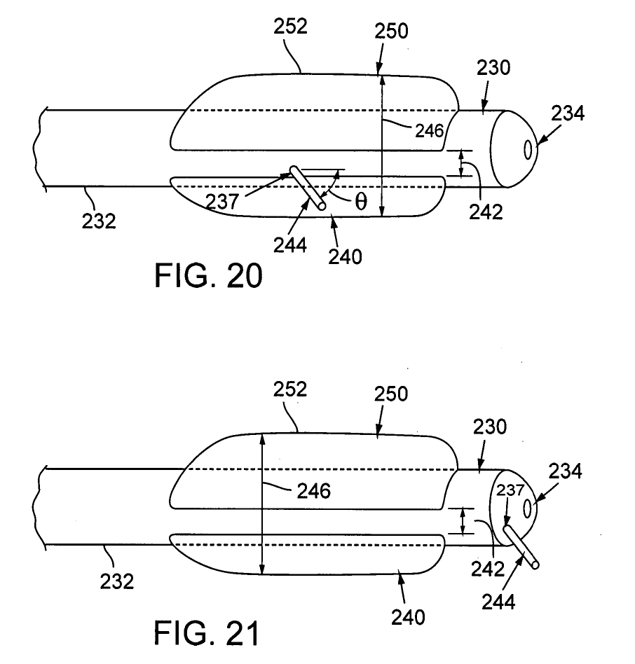

FIG. 19A shows the device of FIG. 18A viewed from the plane defined by arrows 19A of FIG. 18A.

FIG. 20 shows an example of how a piercing tool may extend from the catheter.

FIG. 21 shows another example of how a piercing tool may extend from the catheter.

### DETAILED DESCRIPTION OF THE PREFERRED EMBODIMENTS

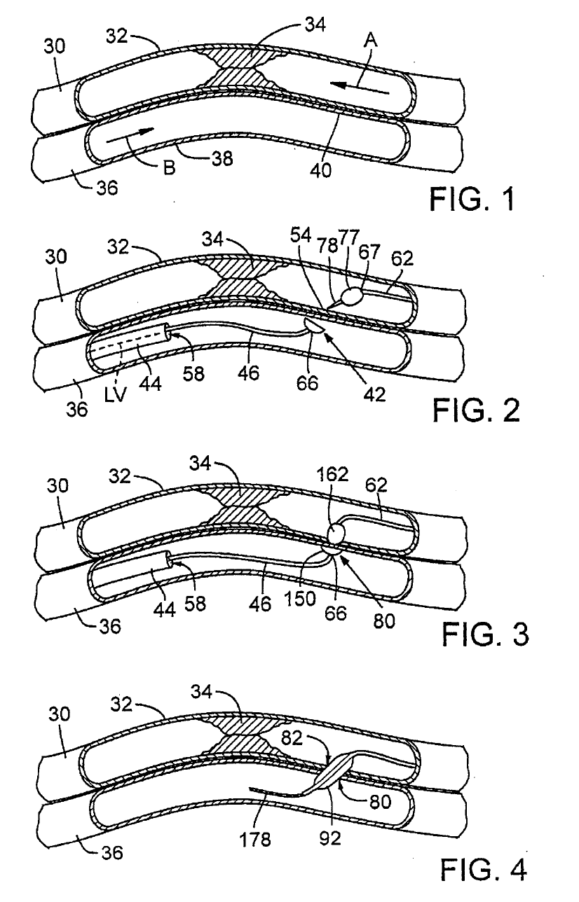

As shown in FIG. 1, an artery 30, formed by an artery wall 32, has a blood flow, indicated by arrow A, that is partially or totally blocked by an obstruction or occlusion 34, typically formed by plaque. A vein 36 roughly similar in dimension to artery 30 lies alongside and generally parallel to artery 30. Vein 36, formed by a vein wall 38, includes, in the area proximal to occlusion 34, a portion 40 in close proximity to artery 30 that the physician has selected as a venous site for creating a fistula between artery 30 and vein 36. The normal blood flow through vein 36 would be in the direction indicated by arrow B.

An embodiment of the disclosed system, indicated generally at 42 in FIG. 2, is a catheter apparatus that includes a first catheter 62 and a second catheter 44. In FIG. 2, the first catheter is in the artery and the second catheter is in the vein, but this can be reversed. Similarly, the first catheter in the artery is shown upstream from occlusion 34, but this may alternatively be reversed to begin the procedure downstream from the occlusion and proceeding afterwards to the upstream side.

Second catheter 44 may include at least one lumen 58 which runs generally parallel to a longitudinal axis LV of catheter 44. A wire 46 may be inserted through lumen 58. Typically, wire 46 has an outer diameter of 0.035-inches, but any suitable dimension may be used. Wire 46 may be controllable by the physician in position relative to catheter 44. Wire 46 may be a guidewire for catheter 44, or a separate guidewire may be used, with other lumens in catheter 44 providing the channel for the separate guidewire.

As shown in FIG. 2, first catheter 62 of catheter apparatus 42 includes a distal end 67 that the physician may insert into artery 30 for positioning adjacent arterial fistula site 54. First catheter 62 may include one or more lumens running generally parallel to a longitudinal axis of catheter 62. First catheter 62 may be guided along a guidewire or may itself be a guidewire, typically with an outer diameter of 0.035-inches, although any suitable dimension may be used. First catheter 62 preferably is hollow.

A piercing tool 77 that includes a sharp needle 78, may be selectively deployed, as shown in FIGS. 2 and 3, or withdrawn into the lumen of catheter 62. Needle 78 is preferably withdrawn while catheter 44 is maneuvered to the fistula site so as not to cause trauma to the blood vessel wall.

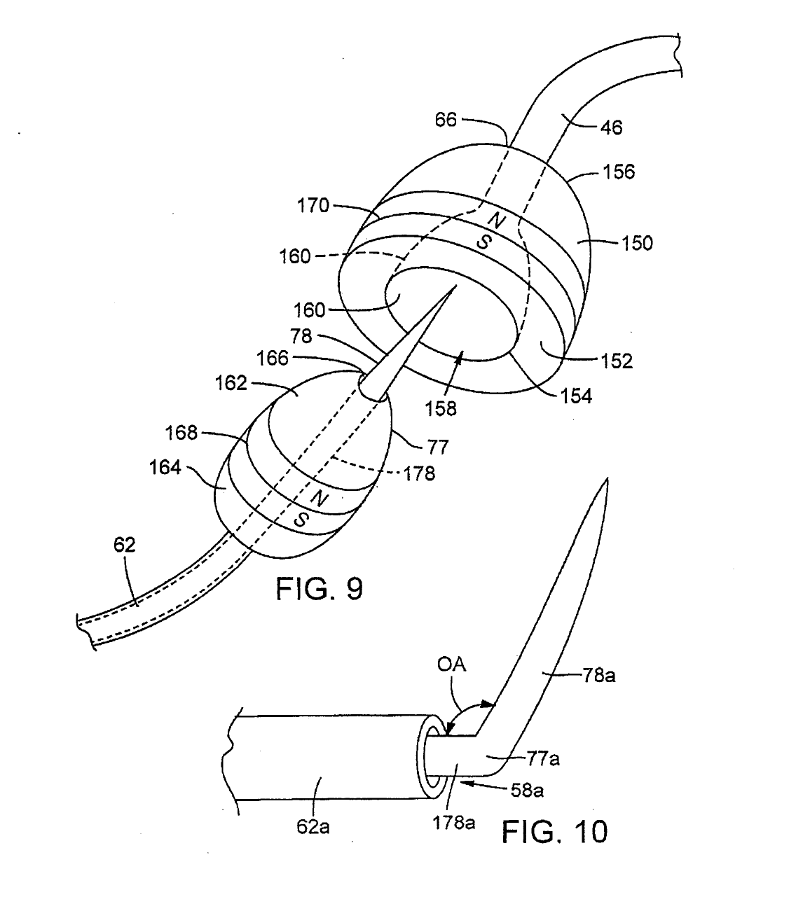

As best seen in FIG. 9, needle 78 may be disposed at the distal end of a wire 178 disposed in the lumen of catheter 62. The physician can control the positioning of wire 178 and needle 78 relative to catheter 62. Guidewire 46 may include a receptor 150, such as substantially cup-shaped socket 152. Receptor 150 includes a distal opening 154, preferably circular, and a proximal end 156. Receptor 150 includes a channel 158 leading from opening 154 toward proximal end 156. Channel 158 preferably narrows in a direction from opening 154 toward proximal end 156. Channel 158 is defined by an inner surface 160 that provides a guide surface for needle 78 that directs the needle toward proximal end 156 of receptor 150. Channel 158 may be substantially conical, or have such other shape as tends to mate with, and guide piercing tool 77 into receptor 150.

Piercing tool 77 on catheter 62 preferably includes a plug 162 provided with an outer contour that narrows from a proximal end 164 toward a distal end 166. Plug 162 preferably mates with channel 158 in receptor 150. Plug 162 preferably encompasses catheter 62 adjacent the distal end of the catheter. As seen in FIGS. 2, 3, and 9, the piercing tool and the receptor have a complementary configuration that supports their mating together.

Typically, piercing tool 77 will include a magnet with one pole oriented toward the distal end of the tool, while receptor 150 will include a magnet with the opposite pole oriented toward the distal end of the receptor which will draw the needle into the receptor. For example, the magnets may be annular rings or donuts and formed of a strong permanent magnet material suitable for the intended use.

A typical arrangement, shown in FIG. 9, is that plug 162 includes a first magnet 168 generally in a donut shape and having a north pole N positioned distally with respect to a south pole S. Typically magnet 168 is spaced from the distal end of plug 162. A second magnet 170 may be disposed on, or form an integral part of receptor 152, preferably adjacent distal opening 154 of socket 152. Second magnet 170 may be arranged with a south pole S distal of a north pole N to attract magnet 168 when the tips of the two catheters are in proximity, e.g., with each catheter in an adjacent blood vessel. Alternatively or in addition one or more magnets may be arranged in various locations on plug 162 and/or needle 78 and on or in receptor 150, e.g., adjacent proximal end 156, with the poles arranged to draw piercing tool 77 into receptor 150.

As shown in FIGS. 3 and 4, after creating openings 80, 82 with a tool such as needle 78, the physician withdraws catheter 62 from the fistula site, leaving wire 178 in place, and a balloon 92 may be inserted over wire 178 and through openings 80, 82 and inflated to enlarge the openings. Balloon 92 may include radiopaque markers and may be inflated with a solution containing a radiopaque dye or contrast to allow the physician to radiographically monitor and adjust the position of the balloon before, during, and after inflation.

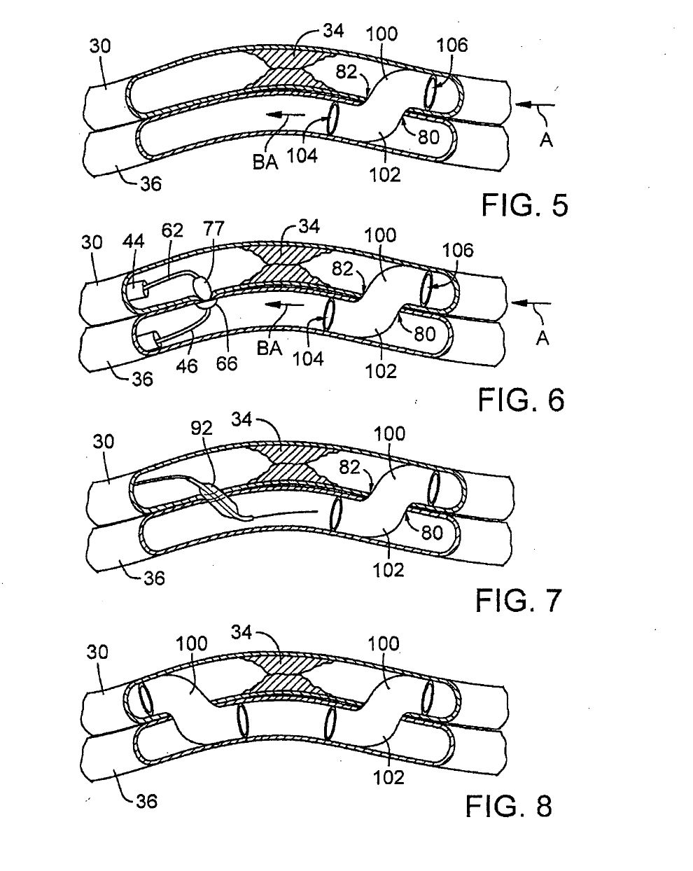

As shown in FIG. 5, a device for maintaining an open, leak-free connection between openings 80 and 82, such as stent 100, is inserted through the openings. Stent 100 includes a frame 102 having two open ends 104 and 106 that preferably create leak-free couplings to the inside of artery 30 and vein 36. With openings 80, 82 connected to form a fistula, vein 36 is arterialized, and blood flows from artery 30 into vein 36 in the direction indicated by arrows A and BA.

Stent 100 is typically a short, covered stent, such as the Hemobahn stent made by WL Gore & Associates.

As shown in FIGS. 6, 7, and 8 a second pair of co-located openings may be created, and a stented fistula established therebetween, using essentially the same catheter system and method as described for FIGS. 1-5 and 9. FIG. 6 illustrates that the first catheter with the piercing tool preferably is inserted into the artery and the openings created from the artery into the vein. Alternatively the openings may be created from the vein into the artery.

An alternative embodiment for the piercing tool is shown in FIG. 10. This tool 77a may be used with a metal guidewire 62a that preferably includes a lumen 58a. An inner wire 178a may be inserted in lumen 58a, providing a base for a needle 78a. The coupling between the needle and base incorporates a curvature such that the needle is nominally offset from the base by an angle OA, typically between about 30-degrees and about 90-degrees. Inner wire 178a is typically made of a sufficiently rigid material, such as nitinol and/or stainless steel, as to maintain the offset angle as the needle is used to pierce blood vessels. Guidewire 62a is preferably formed of a sufficiently rigid material such that when needle 78a is retracted into lumen 58a, the curvature between the needle and the base is overcome and the needle temporarily aligns with the base in a non-traumatic configuration. Inner wire 178a may have an outer diameter of 0.010, 0.014, 0.018, or 0.021-inches, or such other dimension as is suited to the particular application.

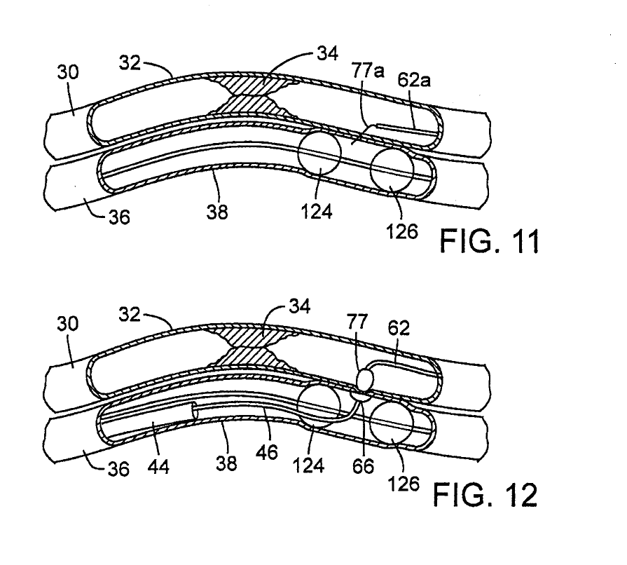

As shown in FIG. 11, piercing tool 77a may be inserted in artery 30, typically while withdrawn into the catheter 62a while maneuvering to the fistula site. Piercing tool 77a may be used in conjunction with a catheter having two balloons 124 and 126 that are inserted in vein 36. In such case, the catheter tips are maneuvered to opposing sides of the proposed fistula site and balloons 124 and 126 are inflated to press the vein wall against the artery wall. Also, fluid may be injected into the sealed-off area to further press the two blood vessel walls together. Then piercing tool 77a is deployed and maneuvered through the artery and then the vein wall to create openings for forming the fistula as for the embodiments described above.

FIG. 11 depicts the piercing tool and the balloon catheter in different vessels. Alternatively, piercing tool 77a may be inserted in the same blood vessel with the balloon catheter. In such an embodiment, the balloons are preferably independently inflatable, and typically the distal balloon 124 is inflated first to stop blood flow. Then, piercing tool 77a is maneuvered to the fistula site in a manner similar to that for the previously described embodiment, typically with the piercing tool withdrawn into the guidewire to the non-traumatic configuration.

With the piercing tool at the fistula site, the proximal balloon 126 is inflated to seal off the fistula site and also to press the vein against the artery. Then, piercing tool 77a is deployed at the end of guidewire 62a and maneuvered by the physician to create the openings from one blood vessel, through both walls, to the other blood vessel.

In either case, piercing tool 77a may be used to create multiple pairs of co-located openings which are then stented to arterialize a portion of the vein to bypass a blockage using a similar method as described above for the embodiment of FIGS. 1-9.

As shown in FIG. 12, the double balloon catheter may also be used in conjunction with the catheters 44 and 62 that include the mating tips. In this embodiment, the double balloon catheter helps to control blood flow at the planned fistula site and to press the blood vessel walls together to assist in the mating of the tips. The fistula creation otherwise proceeds in a similar manner as for the embodiment of FIGS. 1-9.

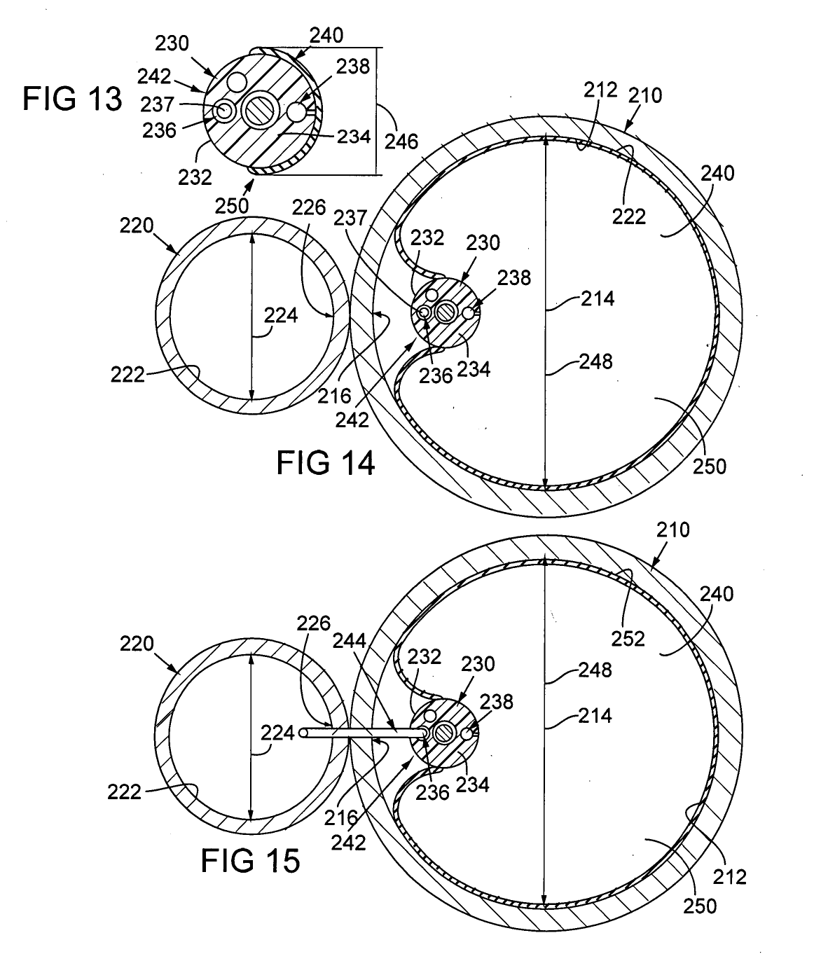

FIG. 13 shows a catheter 230 according to another aspect of the present disclosure, having an outer surface 232, a distal end 234, a first lumen 236 having an opening 237, a second lumen 238, and an resizable portion 240 (shown here as a balloon 250). A piercing tool 244 (not shown) may be contained in the first lumen 236.

The resizable portion 240 may surround any portion of the outer surface 232 of the catheter. In an exemplary embodiment, the resizable portion 240 may surround less than the whole arc of the surface 232. The arc (or arcs) of the surface not surrounded by the resizable portion 240, hereafter known as the free arc(s) 242, may provide an unobstructed pathway for a piercing tool 244 to traverse, as will be further discussed below.

The resizable portion 240 may have a nominal configuration and an active configuration. In the nominal configuration, the resizable portion 240 may have a nominal diameter 246. In the active configuration the resizable portion 240 may have an active diameter 248, which may be greater than the nominal diameter 246. The resizable portion 240 may further be completely or partially radiopaque, so that a physician can view and adjust the position of the free arc 242, and hence control the direction which the piercing tool 244 will fire.

FIG. 14 shows a first vessel 210, formed by a vessel wall 212 and having a first diameter 214, with a first portion 216 that a physician may select as a site for creating an opening for a fistula between first vessel 210 and a second vessel 220. The second vessel 220 lies alongside and generally parallel to first vessel 210. Second vessel 220, formed by a second vessel wall 222 and having a second diameter 224, includes a second portion 226, in the second vessel wall 222 in close proximity to the first portion 216 of the first vessel wall 212, that a physician may select as a site for creating an opening for the fistula from the first vessel 210. An apparatus of the present disclosure, with the resizable portion 240 in the nominal configuration, is seen traversing the first vessel 210.

As seen in FIG. 15, an apparatus of the disclosure is disposed within the first vessel 210 with the resizable portion 240 in the active configuration. In one embodiment, the catheter 230 and resizable portion 240 are rotatable within a vessel. Being rotatable allows the resizable portion 240 to be aligned so that the free arc 242 faces the portion 216 of the first vessel wall 212 intended for a fistula, as seen in FIG. 15, allowing passage of a piercing tool 244. A physician may therefore rotate the apparatus until it is effectively “aimed” at the second blood vessel 220, and fire the piercing tool 244 to create the fistula.

In this particular example, the first vessel diameter 214 is substantially larger than the second vessel diameter 224, which would typically be the case if the first vessel 210 is a vein, and the second vessel 220 is an artery. However, it should be understood any embodiment of the present disclosure may be utilized to create a fistula between any two blood vessels, even where the first diameter 214 may be equal to or less than the second diameter 224.

As mentioned previously, the resizable portion 240 has a nominal configuration and an active configuration. The resizable portion 240 is shown in the active configuration having an active diameter 248 in FIG. 14. The active diameter 248 may be substantially equal to the first diameter 214 of the first vessel 210. It should be understood that having an active diameter 248 substantially equal to the first diameter 214 of the first vessel will cause the resizable portion 240 to stabilize the catheter 230 within the first vessel 210. Some embodiments of the resizable portion 240 may hold the catheter 230 in the center of the first vessel 210. Other embodiments may hold the catheter 230 against a site intended for an opening 216 of a vessel wall 212, or somewhat spaced from such a site 216.

In one example, depicted in FIGS. 13-15, the resizable portion 240 is a balloon 250 having a nominal configuration and an active configuration. In the nominal configuration (seen in FIGS. 13, 20 and 21), the balloon 250 is deflated with a nominal diameter 246 less than the first diameter 214 of the first vessel 210. In the active configuration, the balloon 250 may be inflated to an active diameter 248. The active diameter 248 may be of any size that will hold the catheter 230 stable in the first vessel 210 by causing the balloon surface 252 to contact the wall 212 of the vessel 210, including but not limited to equal to the diameter 214 of the vessel 210, slightly larger than the diameter 214 of vessel 210, or even slightly smaller than the diameter 214 of the first vessel 210.

Balloon 250 may include radiopaque markers and/or may be inflated with a solution containing a radiopaque dye or contrast to allow the physician to radiographically monitor and adjust the position of the balloon 250 before, during, and after inflation. Thus, the Physician may aim the free arc 242 towards the site 216 intended for fistula and the second vessel 220.

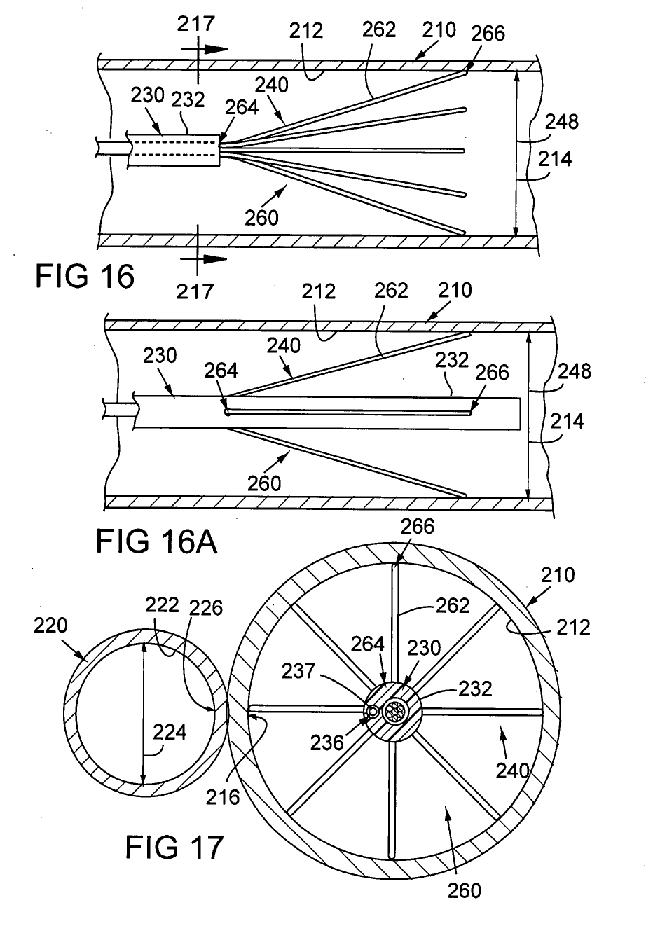

In another embodiment, as shown in FIG. 16, catheter 230 has a resizable portion 240 comprising a plurality of retractable members 260. Each retractable member 262 has a proximal end 264 pivotally coupled to the outer surface 232, and a free distal end 266. In the nominal configuration, each retractable member 262 is retracted so that the distal end 266 is in close proximity with the outer surface 232, allowing for easy movement of the catheter 230 through the first vessel 210. In the active configuration, each retractable member 262 is extended so that the distal end 266 is extended away from the outer surface 232 and abutting the first vessel wall 212.

In some embodiments, some retractable members 260 may be longer than others. In particular, all the retractable members 260 disposed on a particular portion of the outer surface 232 may be longer or shorter than the retractable members 260 disposed on a different portion of the outer surface 232. Such differences in length cause the catheter 230 to be held in a position that is offset from the center of the first vessel 210.

In another embodiment, as shown in FIGS. 18-19 and FIGS. 18A-19A, the resizable portion 240 comprises an expandable stent 270 made of stent cells 272 having an outer surface 274. The stent 270 may be constructed out of any suitable material. In one embodiment, the stent 270 may be metallic. In another embodiment, the stent 270 is at least partially comprised of self-expanding nitinol.

In the nominal configuration, the stent 270 is retracted to a nominal diameter (not shown) which is less than the first diameter 214 of the first vessel 210, allowing for easy movement of the catheter 230 through the first vessel 210. In the active configuration, the stent 270 is expanded to an active diameter 248 substantially equal to the first diameter 214 of the first vessel 210 so that the outer surface 274 of the stent abuts the first vessel wall 212. The active diameter 248 may be of any size that will hold the catheter 230 stable in the vessel 210 by causing the outer stent surface 274 to abut the first wall 212 of the vessel 210, including but not limited to equal to the first diameter 214 of the vessel 210, slightly larger than the first diameter 214, or even slightly smaller than the first diameter 214.

A piercing tool 244 may be selectively deployed, as shown in FIGS. 15, 20 and 21, or withdrawn into the first lumen 236 of catheter 230. The piercing tool 244 is preferably withdrawn while catheter 230 is maneuvered to the fistula site 216 to avoid causing trauma to the blood vessel wall 212. The piercing tool 244 may come in numerous varieties, including but not limited to the instruments recited in U.S. Pat. No. 6,464,665, as well as the piercing tools (77, 77a) described above.

The piercing tool 244 may exit the catheter at the opening 237 of the first lumen. The opening 237 may be disposed on the outer surface 232 of the catheter 230 on a side portion of the catheter, as seen in FIG. 20. In this embodiment, the piercing tool 244 may exit the catheter 230 at an angle θ to the longitudinal pole of the catheter 230. θ may be any angle between 0° and 90°.

The opening 237 may alternatively be disposed on the catheter 230 in the general area of the distal end 234, as seen in FIG. 21. In this embodiment, the piercing tool 244 may exit the catheter 230 at any angle from perpendicular to the longitudinal axis of the catheter 230 to parallel to the longitudinal axis.

It is believed that the following claims particularly point out certain combinations and subcombinations that are directed to one of the disclosed disclosures and are novel and non-obvious. Disclosures embodied in other combinations and subcombinations of features, functions, elements and/or properties may be claimed through amendment of the present claims or presentation of new claims in this or a related application. Such amended or new claims, whether they are directed to a different disclosure or directed to the same disclosure, whether different, broader, narrower or equal in scope to the original claims, are also included within the subject matter of the disclosures of the present disclosure.

We claim:

1. A catheter system for piercing a first wall of a first blood vessel, defined by a first vessel wall and having a first diameter, and a second wall of a second blood vessel to create a fistula between the blood vessels, the system comprising:
a first catheter having an outer surface and a distal end insertable to a position wherein the distal end is adjacent to a site within the first blood vessel for creating the fistula;
a resizable portion for stabilizing the first catheter within the first blood vessel, the resizable portion disposed on the outer surface so that a first arc of the outer surface is unobstructed by the resizable portion, the resizable portion being manipulable between a nominal configuration and an active configuration, the nominal configuration having a diameter less than the first diameter, and the active configuration having a diameter substantially equal to the first diameter; and
a piercing tool for creating the fistula, the piercing tool disposed adjacent the distal end of the catheter and extendable through the first arc of the outer surface.

2. The catheter system of claim 1, wherein the resizable portion is a balloon which is deflated in the nominal configuration and inflated in the active configuration, and the catheter includes a lumen configured to inflate the balloon.

3. The catheter system of claim 2, wherein the balloon is inflated with radiopaque dye.

4. The catheter system of claim 2, the balloon further including radiopaque markers.

5. The catheter system of claim 1, the resizable portion including three or more retractable members, each member having a proximal end pivotally coupled to the outer surface and a free distal end, whereby the distal ends of the members are in close proximity to the outer surface in the nominal configuration and extended away from the outer surface to the first wall of the first blood vessel in the active configuration.

6. The catheter system of claim 5, wherein the members are metallic.

7. The catheter system of claim 5, wherein the members include nitinol.

8. The catheter system of claim 1, wherein the resizable portion comprises an expandable stent whereby the stent in the nominal configuration is constrained and the stent in the active configuration is expanded.

9. The catheter system of claim 8, wherein the stent is metallic.

10. The catheter system of claim 8, wherein the stent includes nitinol.

11. The catheter system of claim 1, wherein the resizable portion is at least partially radiopaque.

12. The catheter system of claim 1 further comprising:
a second catheter having a distal end insertable to a position wherein the distal end is adjacent a site within the second blood vessel for the fistula, the second catheter including adjacent the distal end a receptor having a distal opening and a proximal end, the receptor further including a channel leading from the opening toward the proximal end; and
one or more magnets disposed on at least one of the catheters to draw the piercing tool into the channel of the receptor.

13. The catheter system of claim 12 wherein the channel of the receptor narrows in a direction from the opening toward the proximal end.

14. The catheter system of claim 13 wherein the channel is substantially conical.

15. The catheter system of claim 12 wherein one magnet is disposed at the proximal end of the receptor.

16. The catheter system of claim 12 wherein one magnet is disposed on the first catheter proximally with respect to the piercing tool.

17. The catheter system of claim 16 wherein the magnet on the first catheter encompasses the catheter adjacent the distal end and includes a portion that fits into the channel of the receptor.

18. The catheter system of claim 12, wherein the piercing tool is magnetic.

19. The catheter system of claim 12, wherein the piercing tool and the receptor are provided with a complementary configuration.

20. A catheter system for piercing a first wall of a first blood vessel, defined by a first vessel wall and having a first diameter, and a second wall of a second blood vessel to create a fistula between the blood vessels, the system comprising:
a first catheter having an outer surface, a first lumen, a distal end insertable to a position wherein the distal end is adjacent to a site within the first blood vessel for creating the fistula, and including:
a resizable portion for stabilizing the first catheter within the first blood vessel, the resizable portion disposed on the outer surface so that a first arc of the outer surface is unobstructed by the resizable portion, the resizable portion being manipulable between a nominal configuration and an active configuration, the nominal configuration having a diameter less than the first diameter, and the active configuration having a diameter substantially equal to the first diameter; and
a piercing tool for creating the fistula, the piercing tool disposed adjacent the distal end of the catheter and extendable through the first arc of the outer surface; and
a second catheter having a distal end insertable to a position wherein the distal end is adjacent to a site within the second blood vessel for the fistula, the second catheter including adjacent to the distal end a receptor having a distal opening, proximal end, and a guide surface disposed between the distal opening and the proximal end; and
one or more magnets disposed on at least one of the catheters to draw the piercing tool along the guide surface of the receptor.

21. The catheter system of claim 20 wherein the guide surface of the receptor directs the piercing tool toward the proximal end.

22. The catheter system of claim 20 wherein the guide surface includes a conical section.

23. The catheter system of claim 20 wherein one magnet is disposed at the proximal end of the receptor.

24. The catheter system of claim 20 wherein one magnet is disposed on the first catheter proximally with respect to the piercing tool.

25. The catheter system of claim 24 wherein the magnet on the first catheter encompasses the catheter adjacent the distal end and includes a portion that mates with the guide surface of the receptor.

26. The catheter system of claim 20 wherein the piercing tool is magnetic.

27. The catheter system of claim 20 wherein the piercing tool and the receptor are provided with a complementary configuration.

28. The catheter system of claim 20, wherein the resizable portion is a balloon which is deflated in the nominal configuration and inflated in the active configuration, and the first catheter includes a lumen configured to inflate the balloon.

29. The catheter system of claim 28, wherein the balloon is inflated with radiopaque dye.

30. The catheter system of claim 28, the balloon further including radiopaque markers.

31. The catheter system of claim 20, the resizable portion including three or more retractable members, each member having a proximal end pivotally coupled to the outer surface and a free distal end, whereby the distal ends of the members are in close proximity to the outer surface in the nominal configuration and extended away from the outer surface to the first wall of the first blood vessel in the active configuration.

32. The catheter system of claim 31, wherein the members are metallic.

33. The catheter system of claim 31, wherein the members include nitinol.

34. The catheter system of claim 20, wherein the resizable portion comprises an expandable stent whereby the stent in the nominal configuration is constrained and the stent in the active configuration is expanded.

35. The catheter system of claim 34, wherein the stent is metallic.

36. The catheter system of claim 34, wherein the stent includes nitinol.

37. The catheter system of claim 20, wherein the resizable portion is at least partially radiopaque.
